(19) United States
(12) Patent Application Publication
(10) Pub. No.: US 2015/0190523 A1
(43) Pub. Date: Jul. 9, 2015

(54) BIOAVAILABILITY OF ORAL METHYLNALTREXONE INCREASES WITH A PHOSPHATIDYLCHOLINE-BASED FORMULATION

(71) Applicant: The University of Chicago, Chicago, IL (US)

(72) Inventors: Chun-Su Yuan, Chicago, IL (US); Chong-Zhi Wang, Chicago, IL (US); Maojian Gu, Chicago, IL (US); Dong-Hai Lin, Chicago, IL (US)

(21) Appl. No.: 14/398,914

(22) PCT Filed: Mar. 13, 2013
(86) PCT No.: PCT/US13/31078

§ 371 Date: Nov. 4, 2014

Related U.S. Application Data
(60) Provisional application No. 61/642,837, filed on May 4, 2012.

(51) Int. Cl. A61K 47/48; A61K 31/485

(52) U.S. Cl. CPC A61K 47/48053; A61K 31/485

(57) ABSTRACT

A pharmaceutical composition comprising a phosphatidylcholine-based opioid receptor antagonist formulation, as well as methods of their use and methods of their preparation are provided herein. Such pharmaceutical composition may be used for treating and preventing opioid-induced side effects in patients, and may be provided to chronic opioid users as well.

This application claims priority to U.S. Provisional Application 61/642,837 filed on May 4, 2012, which is hereby incorporated by reference in its entirety.

### BACKGROUND

1. Field of the Invention

The present invention relates to the fields of opioid receptor antagonists and drug delivery. In general, compositions comprising an opioid receptor antagonist formulation are described along with methods of their use.

2. Description of Related Art

Opioid medications are widely used as analgesics to treat moderate to severe pain in both cancer and non-cancer patients. Although opioids are effective in managing pain, their use is associated with a number of undesirable side effects. One most common and distressing side effect is opioid-induced gastrointestinal dysfunction (Mehendale and Yuan, 2006; Warfield, 1998). Patients who receive chronic opioid treatment frequently experience constipation (Glare and Lickiss, 1992; Mehendale and Yuan, 2006). Opioid-induced constipation is found in 90% of patients treated with opioids and is a significant problem in 40%-45% of patients with advanced cancer (Walsh, 1984; Yap and Pappagallo, 2005). Treatment of constipation routinely involves the regular use of various laxatives which often do not provide sufficient relief to patients with opioid-induced constipation (Mehendale and Yuan, 2006; Kurz and Sessler, 2003) and causes side effects such as electrolyte imbalances. In severe cases, patients may choose to limit or discontinue opioids to reduce the discomfort of bowel dysfunction.

One treatment for opioid side effects is the use of opioid receptor antagonists which cross the blood-brain-barrier, or which are administered directly into the central nervous system. Opioid receptor antagonists such as naltrexone and naloxone have been administered intramuscularly or orally to treat opioid induced side effects. Naltrexone and naloxone are highly lipid soluble and rapidly diffuse across biological membranes, including the blood-brain barrier. However, naltrexone, naloxone, nalmefene, and other opioid receptor antagonists which may reverse many opioid side effects have a narrow therapeutic window before they are observed to reverse the desired analgesic effect of the opioid being used, thus their utility in cancer patients with chronic opioid constipation is greatly limited (Klepstad et al., 2000; Mercadante et al., 2000).

Another treatment for opioid side effects is the use of quaternary amine opioid receptor antagonist derivatives, such as methylnaltrexone (MNTX). MNTX, a quaternary derivative of naltrexone, is a peripherally acting, mu-opioid receptor-selective antagonist of opioid action in tissues (Yuan, 2007; Thomas et al., 2008). MNTX is formed by the addition of a methyl group at the amine ring of naltrexone, and has a positive charge in solution. As a result, MNTX has greater polarity and lower lipid solubility than other clinically used opioid receptor antagonists. Because of these characteristics, MNTX does not cross the blood-brain barrier and functions as a peripherally acting opioid receptor antagonist in the gastrointestinal tract where it decreases constipation without impacting centrally mediated analgesia (Yuan, 2007; Russell et al., 1982; Brown and Goldberg, 1985). Thus, MNTX offers therapeutic potential to prevent or treat chronic opioid-induced constipation and improve the quality of life in cancer patients (Yuan, 2007; Osinski et al., 2002).

In April 2008, the United States FDA approved the use of methylnaltrexone bromide (RELISTOR®) as a subcutaneous injection to help restore bowel function in patients with late-stage, advanced illness who are receiving opioids on a continuous basis to help alleviate their pain (Michna et al., 2011; Rotshteyn et al., 2011). In particular, the drug is designed to alleviate constipation in patients who have not successfully responded to laxative therapy. The injectable form of MNTX bromide (RELISTOR®), has also been approved in Europe and many other countries for the treatment of opioid-induced constipation in patients when response to laxative therapy has not been sufficient (Michna et al., 2011; FDA/CDER, 2008).

As a subcutaneous injection, MNTX is administered in a single dose every other day, as needed, but no more frequently than one dose in a 24-hour period. Compared to a subcutaneous injection, oral administration is a more convenient, economic, and safer method for drug delivery. As a hydrophilic compound, MNTX has limited gastrointestinal absorption, i.e., a low oral bioavailability (Yuan et al., 1997; Yuan and Foss, 2000).

Therefore, alternative formulations and methods of providing MNTX and other opioid receptor antagonists are desirable, such as formulations that increase the bioavailability of the antagonists, and methods less intrusive than subcutaneous injection.

### SUMMARY OF THE INVENTION

The present invention provides methods and compositions comprising an opioid receptor antagonist formulation. In some embodiments, an opioid receptor antagonist is formulated with phosphatidylcholine (PC). In some embodiments, these compositions allow for preventing or treating opioid-induced bowel dysfunction and other indications. For example, the compositions of the present invention may result in improved absorption of the opioid receptor antagonist into the circulatory system compared to traditional formulations, or formulations comprising an opioid receptor antagonist, thus resulting in a decrease in the dose required to reach therapeutic plasma levels. The compositions may also be employed in preventative methods as well, such as to prevent opioid-induced side effects. Moreover, the opioid responsible for the opioid-induced effects may be an exogenously administered opioid, or an endogenous opioid that is produced by a patient in response to, for example, abdominal surgery. Chronic opioid users may also benefit from receiving the compositions of the present invention.

Accordingly, embodiments relate to a pharmaceutical composition comprising an opioid receptor antagonist formulation. In certain embodiments, the composition comprises an opioid receptor antagonist formulated with phosphatidylcholine (PC).

An opioid receptor antagonist that is formulated as disclosed herein may be, for example, a peripheral opioid antagonist. In certain embodiments, the opioid receptor antagonist may be a quaternary or tertiary morphinan derivative, a piperidine-N-alkylcarboxylate, a carboxy-normorphinan derivative, or a quaternary benzomorphan. The quaternary morphinan may be, for example, a quaternary salt of N-methylnaltrexone, N-methylnaloxone, N-methylnalorphine, N-diallylnormorphine, N-allyllevellorphan, or N-methylnalmefene. In certain embodiments, the peripheral opioid receptor antagonist that is formulated is methylnaltrexone (MNTX). In some embodiments, the opioid receptor antagonist formulation is methylnaltrexone (MNTX) formulated with phosphatidylcholine (PC) (MNTX-PC).

In some embodiments, a pharmaceutical composition may comprise one or more opioid receptor antagonist formulations. In certain embodiments, the weight percentage of total opioid receptor antagonist formulations in the composition ranges from about, at most about, or at least about 0.1-30%. In certain embodiments, the weight percentage of total opioid receptor antagonist formulations is about 0.1%, 0.25%, 0.5%, 0.75%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%, or any range derivable therein. The weight percentage of total opioid receptor antagonist formulations in the particle may range higher than 30%, in certain embodiments. In certain embodiments, the weight percentage may be about, at least about, or at most about 35%, 40%, 45%, 50%, 55%, 60%, 65%, 70%, 75%, 80%, 85%, 90%, 95%, or 99%, or any range derivable therein. In some embodiments, a pharmaceutical composition may also comprise one or more opioid receptor antagonist formulations in combination with one or more opioid receptor antagonists.

Pharmaceutical compositions typically comprise at least one pharmaceutically acceptable carrier. The pharmaceutical composition may be further defined as an orally administrable pharmaceutical composition. The orally administrable pharmaceutical composition may, in certain embodiments, be comprised in a suspension or capsule. The orally administrable pharmaceutical composition may further comprise a flavoring agent. The pharmaceutical composition of the present invention may be further defined as a time release pharmaceutical composition, wherein the time release pharmaceutical composition is formulated to release the opioid receptor antagonist formulation over time.

Methods of making phosphatidylcholine (PC)-based opioid receptor antagonist are also contemplated. The method may comprise, for example: (a) dissolving an opioid receptor antagonist and phosphatidylcholine (PC) in a solvent to form a mixture; (b) heating the mixture; (c) removing the solvent to obtain a residual; and (d) lyophilizing the residual to form a solid substance of phosphatidylcholine (PC)-based opioid receptor antagonist. Optionally, the method may comprise, prior to lyophilization, (i) dissolving the residual in a second solvent, and optionally (ii) removal of the second solvent to obtain a second residual. The method may also comprise, for example: (u) dissolving an opioid receptor antagonist and phosphatidylcholine (PC) in a first solvent to form a mixture; (v) heating the mixture; (w) removing the first solvent to obtain a first residual; and (x) dissolving the first residual in a second solvent, (y) removing the second solvent to create a second residual, and (z) lyophilizing the second residual to form a solid substance of phosphatidylcholine (PC)-based opioid receptor antagonist. In some embodiments, the method comprises: (l) dissolving an opioid receptor antagonist and phosphatidylcholine (PC) in a first solvent to form a mixture; (m) heating the mixture; (n) removing the first solvent to obtain a first residual; (o) dissolving the first residual in a second solvent, (p) filtering the second solvent to remove the second residual and obtain a filtrate (q) removing the second solvent from the filtrate to obtain a third residual, and (r) lyophilizing the third residual to form a solid substance of phosphatidylcholine (PC)-based opioid receptor antagonist.

The opioid receptor antagonist may be any opioid receptor antagonist described herein. In some embodiments, the opioid receptor antagonist is methylnaltrexone (MNTX). In certain embodiments, the solvent or solvents may be methanol, ethanol, tetrahydrofuran, or chloroform. In some embodiments, the solvent is ethanol. In some embodiments, the opioid receptor antagonist is MNTX and the solvent is ethanol. In some embodiments, the first solvent is ethanol and the second solvent is chloroform. In some embodiments, the first solvent is ethanol, the second solvent is chloroform, and the opioid receptor antagonist is MNTX. In some embodiments, the molar ratio between the opioid receptor antagonist and phosphatidylcholine (PC) is from 2:1 to 1:10, preferably from 1:1 to 1:5, more preferably 1:2.

Methods of administering the pharmaceutical compositions of the present invention are also contemplated, and such methods are described herein. For example, the method can comprise administering a pharmaceutical composition comprising an opioid receptor antagonist formulation and a pharmaceutically acceptable carrier to a patient. Any opioid receptor antagonist formulation of the present invention may be employed in such methods. In certain embodiments, the opioid receptor antagonist formulation is an opioid receptor antagonist formulated with phosphatidylcholine (PC). In some embodiments, the opioid receptor antagonist formulation is methylnaltrexone-based. In some embodiments, the methylnaltrexone is formulated with phosphatidylcholine (MNTX-PC).

As discussed herein, such administration may be, e.g., orally, intraadiposally, intraarterially, intraarticularly, intradermally, intralesionally, intramuscularly, intranasally, intraocularally, intraperitoneally, intrapleurally, intrarectally, intrathecally, intratracheally, intraumbilically, intravenously, intravesicularly, intravitreally, liposomally, locally, mucosally, parenterally, rectally, subconjunctival, subcutaneously, sublingually, topically, transbuccally, transdermally, in creams, in lipid compositions, via a catheter, via a lavage, via continuous infusion, via infusion, via inhalation, via injection, via local delivery, via localized perfusion, bathing target cells directly, or any combination thereof. In some embodiments, the administration is orally, intravenously, or via injection. In some embodiments, the administration is orally.

Dosages of the pharmaceutical compositions of the present invention are described herein. In certain embodiments of any method described herein, the dosage of a composition comprising an opioid receptor antagonist formulation such as MNTX-PC, is about 0.1-100.0 mg/kg body weight, preferably 0.5-50.0 mg/kg, more preferably, 2.0 mg/kg body weight, or other ranges as described herein.

Patients or subjects of any appropriate method described herein are described below. For example, a patient may be suffering from or may be at risk of suffering from constipation, dysphoria, pruritus, or urinary retention. In certain embodiments, the patient is suffering from or is at risk of suffering a disorder selected from ileus, post-operative ileus, paralytic ileus, post-partum ileus, gastrointestinal dysfunction developing following abdominal surgery, and idiopathic constipation. In certain embodiments, the patient is suffering from a disorder mediated by opioid receptor activity selected from cancer involving angiogenesis, an inflammatory disorder, immune suppression, a cardiovascular disorder, chronic inflammation, chronic pain, sickle cell anemia, a vascular wound, retinopathy, decreased biliary secretion, decreased pancreatic secretion, biliary spasm, and increased gastroesophageal reflux.

Other general aspects are directed to a method for preventing an opioid-induced side effect in a patient comprising orally administering an effective amount of a pharmaceutical composition of the present invention comprising an opioid receptor antagonist formulation, such as MNTX-PC, and a pharmaceutically acceptable carrier to the patient prior to, or at the same time of administration of an opioid, The opioid induced side effect may comprise, for example, at least one effect selected from inhibition of intestinal motility, gastrointestinal dysfunction, constipation, bowel hypomotility, impaction, gastric hypomotility, inhibition of gastric motility, inhibition of gastric emptying, delayed gastric emptying, incomplete evacuation, nausea, emesis, cutaneous flushing, bloating, abdominal distension, sweating, dysphoria, pruritis, and urinary retention.

Also contemplated are methods for treating an opioid induced side effect comprising administering, e.g., orally administering, an effective amount of a pharmaceutical composition of the present invention comprising an opioid receptor antagonist formulation, such as MNTX-PC, to a patient subsequent to administration of an opioid.

A subject who suffers from an opioid-induced side effect may suffer from a side effect arising from opioid therapy with, for example, alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine. (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and/or tramadol.

Methods for treating gastrointestinal dysfunction following abdominal surgery comprising administering a pharmaceutical composition of the present invention to a patient are contemplated, comprising orally administering an effective amount of a composition comprising an opioid receptor antagonist formulation, such as MNTX-PC to a patient, wherein the dysfunction is treated.

Methods for preventing inhibition of gastrointestinal motility in a patient are also contemplated, such as methods for preventing inhibition of gastrointestinal motility in a patient prior to the patient receiving an opioid for pain resulting from surgery comprising administering an effective amount of a pharmaceutical composition of the present invention comprising an opioid receptor antagonist formulation, such as MNTX-PC, to the patient.

Another general aspect is directed to a method for treating inhibition of gastrointestinal motility in a patient receiving an opioid for pain resulting from surgery comprising administering an effective amount of a pharmaceutical composition of the present invention, comprising an opioid receptor antagonist formulation, such as MNTX-PC, to the patient.

Also contemplated are methods of preventing or treating an opioid-induced side effect in a chronic opioid patient, comprising administering an effective amount of a pharmaceutical composition of the present invention, comprising an opioid receptor antagonist formulation, such as MNTX-PC, to the patient. The side effect may be, for example, inhibition of intestinal motility, gastrointestinal dysfunction, constipation, bowel hypomotility, impaction, gastric hypomotility, inhibition of gastric motility, inhibition of gastric emptying, delayed gastric emptying, incomplete evacuation, nausea, emesis, cutaneous flushing, bloating, abdominal distension, sweating, dysphoria, pruritis, or urinary retention.

Another aspect relates to a method for increasing gastrointestinal absorption of methylnaltrexone (MNTX) in a patient following oral administration, comprising orally administering to said patient an effective amount of a pharmaceutical composition comprising MNTX-PC to said patient.

Any embodiment discussed with respect to one aspect can apply to other aspects of other embodiments disclosed herein as well.

The embodiments in the Example section are understood to be embodiments that are applicable to all aspects of the methods and compositions disclosed herein.

The term “effective,” as that term is used in the specification and/or claims, means adequate to accomplish a desired, expected, or intended result.

“Therapeutically effective amount” means that amount which, when administered to a subject for treating a condition, disease, or side effect, is sufficient to effect such treatment for the condition, disease, or side effect.

“Treatment” or “treating” includes: (1) inhibiting a condition, disease, or side effect in a subject or patient experiencing or displaying the pathology or symptomatology of the condition, disease, or side effect (e.g., arresting further development of the pathology and/or symptomatology), (2) ameliorating a condition, disease, or side effect in a subject or patient that is experiencing or displaying the pathology or symptomatology of the condition, disease, or side effect (e.g., reversing the pathology and/or symptomatology), and/or (3) effecting any measurable decrease in a condition, disease, or side effect in a subject or patient that is experiencing or displaying the pathology or symptomatology of the condition, disease, or side effect.

“Prevention” or “preventing” includes: (1) inhibiting the onset of a condition, disease, or side effect in a subject or patient who may be at risk and/or predisposed to the condition, disease, or side effect but does not yet experience or display any or all of the pathology or symptomatology of the condition, disease, or side effect, and/or (2) slowing the onset of the pathology or symptomatology of the condition, disease, or side effect in a subject or patient which may be at risk and/or predisposed to the condition, disease, or side effect but does not yet experience or display any or all of the pathology or symptomatology of the condition, disease, or side effect.

As used herein, the term “patient” or “subject” refers to a living mammalian organism, such as a human, monkey, cow, sheep, goat, dog, cat, mouse, rat, guinea pig, or transgenic species thereof. Non-limiting examples of human subjects are adults, juveniles, children, infants and fetuses.

In certain embodiments, a patient is a chronic opioid user. Accordingly, embodiments are useful to prevent or reduce the occurrence or reoccurrence of an opioid-induced side effect in a chronic opioid patient. A chronic opioid patient may be any of the following: a cancer patient, an AIDS patient, or any other terminally ill patient. A chronic opioid patient may be a patient taking methadone. Chronic opioid use is characterized by the need for substantially higher levels of opioid to produce the therapeutic benefit as a result of prior opioid use, as is well known in the art. Chronic opioid use is also characterized by the need for substantially lower levels of opioid antagonist to produce the therapeutic benefit. Chronic opioid use as used herein includes daily opioid treatment for a week or more or intermittent opioid use for at least two weeks. In some embodiments, a patient, such as a chronic opioid user, is taking a laxative and/or a stool softener.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Accordingly, pharmaceutically acceptable salts of compounds are contemplated herein. Such pharmaceutically acceptable salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

It is contemplated that any embodiment discussed in this specification can be implemented with respect to any method or composition as disclosed herein, and vice versa. Furthermore, compositions as disclosed herein can be used to achieve the methods described herein.

It is also contemplated that any method described herein may be described using Swiss-type use language.

The use of the term “or” in the claims is used to mean “and/or” unless explicitly indicated to refer to alternatives only or the alternatives are mutually exclusive.

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The terms “comprise,” “have” and “include” are open-ended linking verbs. Any forms or tenses of one or more of these verbs, such as “comprises.” “comprising,” “has,” “having,” “includes” and “including,” are also open-ended. For example, any method that “comprises,” “has” or “includes” one or more steps is not limited to possessing only those one or more steps and also covers other unlisted steps.

Other objects, features and advantages of embodiments described herein will become apparent from the following detailed description. It should be understood, however, that the detailed description and the specific examples, while indicating specific embodiments, are given by way of illustration only, since various changes and modifications within the spirit and scope of the embodiments disclosed herein will become apparent to those skilled in the art from this detailed description.

### BRIEF DESCRIPTION OF THE DRAWINGS

The following drawings form part of the present specification and are included to further demonstrate certain aspects as disclosed herein. Embodiments may be better understood by reference to one or more of these drawings in combination with the detailed description of specific embodiments presented herein.

FIG. 1 shows chemical structures of methylnaltrexone bromide (MNTX) and ketamine hydrochloride, the internal standard.

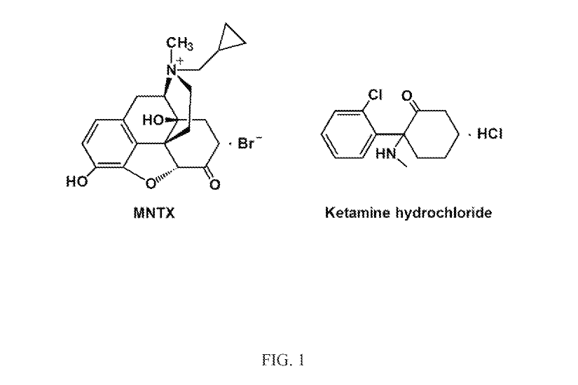

FIG. 2 shows UV spectra of PC, MNTX and MNTX-PC.

FIGS. 3A-3D show X-ray diffraction patterns of MNTX (A), PC (B), physical mixture of MNTX and PC (C), and MNTX-PC (D).

FIGS. 4A-4B show ESI-MS spectra of MNTX (A) and ketamine hydrochloride, the internal standard (B).

FIG. 5 shows MNTX plasma concentrations at the indicated times after oral administration of 250 mg/kg MNTX water solution or 250 mg/kg MNTX-PC in rats. Each value represents the mean±standard error (n=5).

### DESCRIPTION OF ILLUSTRATIVE EMBODIMENTS

The present invention is based, at least in part, on the finding that particular formulations of opioid receptor antagonists, when complexed with phosphatidylcholine (PC), exhibit enhanced stability and, further, result in unexpectedly enhanced bioavailability of the opioid antagonist. In particular, a pharmaceutical composition including methylnaltrexone complexed with phosphatidylcholine has been shown to dramatically increase the bioavailability of methylnaltrexone upon administration, to an extent not predictable based on prior formulations of methylnaltrexone. Moreover, such formulations have been shown exhibited particular stability. In view of these findings, the present invention provides improved methylnaltrexone pharmaceutical compositions, for example, oral compositions, that achieve therapeutic efficacy in, for example, preventing or treating opioid-induced bowel dysfunction such as constipation, at reduced levels of methylnaltrexone, as compared to existing formulations.

### Opioid Receptor Antagonists

Embodiments encompass opioid receptor antagonists formulations, in particular, opioid receptor antagonists formulated with phosphatidylcholine (PC). Any opioid receptor antagonist described herein may be used to form opioid receptor antagonist formulations contemplated and disclosed herein. The opioid receptor antagonists that are formulated include both centrally and peripherally acting opioid receptor antagonists. In certain embodiments, formulations comprising peripherally acting opioid receptor antagonists are contemplated.

Opioid receptor antagonists form a class of compounds that can vary in structure while maintaining their antagonist properties. These compounds include tertiary and quaternary morphinans, such as noroxymorphone derivatives; N-substituted piperidines, such as piperidine-N-alkylcarboxylates, tertiary and quaternary benzomorphans, and tertiary and quaternary normorphinan derivatives, such as 6-carboxy-normorphinan derivatives. Tertiary compound antagonists are fairly lipid soluble and cross the blood-brain barrier easily. Examples of opioid receptor antagonists that cross the blood-brain barrier and are centrally (and peripherally) active include, e.g., naloxone, naltrexone (each of which is commercially available from Baxter Pharmaceutical Products, Inc.), and nalmefene (available, e.g., from DuPont Pharma). Peripherally restricted antagonists, on the other hand, are typically charged, polar, and/or of high molecular weight; these properties typically impede their crossing the blood-brain barrier. Methylnaltrexone is a quaternary derivative of the tertiary opioid receptor antagonist, naltrexone. Addition of the methyl group to naltrexone forms a compound with greater polarity and lower lipid solubility. Thus, methylnaltrexone does not cross the blood-brain barrier and has the potential for blocking the undesired adverse effects which are typically mediated by peripherally located receptors.

A peripheral opioid receptor antagonist suitable for use in the invention may be a compound which is a quaternary morphinan derivative, such as a quaternary noroxymorphone of formula (I):

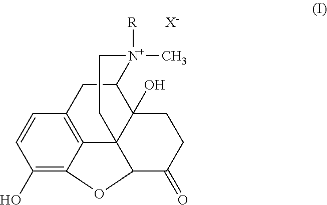

wherein R is alkyl, alkenyl, alkynyl, aryl, cycloalkyl-substituted alkyl, or aryl substituted alkyl, and X⁻ is the anion, such as a chloride, bromide, iodide, or methylsulfate anion. The noroxymorphone derivatives of formula (I) can be prepared, for example, according to the procedure in U.S. Pat. No. 4,176,186, which is incorporated herein by reference; see also U.S. Pat. Nos. 4,719,215; 4,861,781; 5,102,887; 5,972,954; and 6,274,591; U.S. Patent Application Nos. 2002/0028825 and 2003/0022909; and PCT publication Nos. WO 99/22737 and WO 98/25613, each of which is hereby incorporated by reference in its entirety.

A compound of formula (I) may be N-methylnaltrexone (or simply methylnaltrexone), wherein R is cyclopropylmethyl as represented in formula (II):

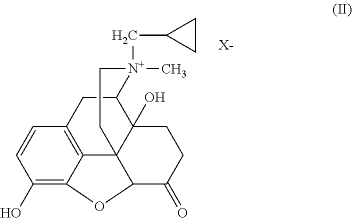

wherein X⁻ may be any pharmaceutically acceptable anion. Methylnaltrexone is a quaternary derivative of the μ-opioid receptor antagonist naltrexone. Methylnaltrexone exists as a salt (e.g., N-methylnaltrexone bromide) and the terms “methylnaltrexone” or “MNTX”, as used herein, therefore embrace such salts. “Methylnaltrexone” or “MNTX” thus specifically includes, but is not limited to, bromide salts, chloride salts, iodide salts, carbonate salts, and sulfate salts of methylnaltrexone. Names used for the bromide salt of MNTX in the literature, for example, include: methylnaltrexone bromide; N-methylnaltrexone bromide; naltrexone methobromide; naltrexone methyl bromide; SC-37359; MRZ-2663-BR; and N-cyclopropylmethylnoroxy-morphine-methobromide. A compound of formula (I) may be S—N-methylnaltrexone.

Methylnaltrexone is commercially available from, e.g., Mallinckrodt Pharmaceuticals, St. Louis, Mo. Methylnaltrexone is provided as a white crystalline powder, freely soluble in water, typically as the bromide salt. The compound as provided is 99.4% pure by reverse phase HPLC, and contains less than 0.011% unquaternized naltrexone by the same method. Methylnaltrexone can be prepared as a sterile solution at a concentration of, e.g., about 5 mg/mL.

Other suitable peripheral opioid receptor antagonists may include, for example, N-substituted piperidines, such as piperidine-N-alkylcarboxylates as represented by formula (III):

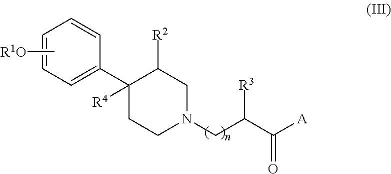

wherein R¹ is hydrogen or alkyl; R² is hydrogen, alkyl, or alkenyl; R³ is hydrogen, alkyl, alkenyl, aryl, cycloalkyl, cycloalkenyl, cycloalkyl-substituted alkyl, cycloalkenyl-substituted alkyl, or aryl-substituted alkyl; R⁴ is hydrogen, alkyl, or alkenyl; A is OR⁵ or NR⁶R⁷; wherein R⁵ is hydrogen, alkyl, alkenyl, cycloalkyl, cycloalkenyl, cycloalkyl-substituted alkyl, cycloalkenyl-substituted alkyl, or aryl-substituted alkyl; R⁶ is hydrogen or alkyl; R⁷ is hydrogen, alkyl, alkenyl, aryl, cycloalkyl, cycloalkenyl, cycloalkyl-substituted alkyl, cycloalkenyl-substituted alkyl or aryl-substituted alkyl, or alkylene-substituted B or together with the nitrogen atom to which they are attached, R⁶ and R⁷ form a heterocyclic ring selected from pyrrole and piperidine; B is

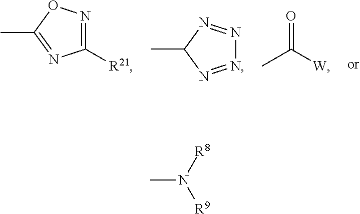

wherein R⁸ is hydrogen or alkyl; R⁹ is hydrogen, alkyl, alkenyl, aryl, cycloalkyl, cycloalkenyl, cycloalkyl-substituted alkyl, cycloalkenyl-substituted alkyl or aryl-substituted alkyl or together with the nitrogen atom to which they are attached, R⁸ and R⁹ form a heterocyclic ring selected from pyrrole and piperidine; W is OR¹⁰, NR¹¹R¹², or OE; wherein R¹⁰ is hydrogen, alkyl, alkenyl, cycloalkyl, cycloalkenyl, cycloalkyl-substituted alkyl, cycloalkenyl-substituted alkenyl, or aryl-substituted alkyl; R¹¹ is hydrogen or alkyl; R¹² is hydrogen, alkyl, alkenyl, aryl, cycloalkyl, cycloalkenyl, cycloalkyl-substituted alkyl, cycloalkenyl-substituted alkyl, aryl-substituted alkyl, or alkylene-substituted C(═O)Y or, together with the nitrogen atom to which they are attached, R¹¹ and R¹² form a heterocyclic ring selected from pyrrole and piperidine; E is

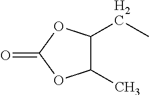

alkylene-substituted (C═O)D, or —R¹³OC(═O)R¹⁴; wherein R¹³ is alkyl-substituted alkylene; R¹⁴ is alkyl; D is OR¹⁵ or NR¹⁶R¹⁷; wherein R¹⁵ is hydrogen, alkyl, alkenyl, cycloalkyl, cycloalkenyl, cycloalkyl-substituted alkyl, cycloalkenyl substituted alkyl, or aryl-substituted alkyl; R¹⁶ is hydrogen, alkyl, alkenyl, aryl, aryl-substituted alkyl, cycloalkyl, cycloalkenyl, cycloalkyl substituted alkyl, or cycloalkenyl-substituted alkyl; R¹⁷ is hydrogen or alkyl or, together with the nitrogen atom to which they are attached, R¹⁶ and R¹⁷ form a heterocyclic ring selected from the group consisting of pyrrole or piperidine; Y is OR¹⁸ or NR¹⁹R²⁰; wherein R¹⁸ is hydrogen, alkyl, alkenyl, cycloalkyl, cycloalkenyl, cycloalkyl-substituted alkyl, cycloalkenyl-substituted alkyl, or aryl-substituted alkyl; R¹⁹ is hydrogen or alkyl; R²⁰ is hydrogen, alkyl, alkenyl, aryl, cycloalkyl, cycloalkenyl, cycloalkyl-substituted alkyl, cycloalkenyl-substituted alkyl, or aryl-substituted alkyl or, together with the nitrogen atom to which they are attached, R¹⁹ and R²⁰ form a heterocyclic ring selected from pyrrole and piperidine; R²¹ is hydrogen or alkyl; and n is 0 to 4.

Non-limiting examples of suitable N-substituted piperidines may be prepared as disclosed in U.S. Pat. Nos. 5,270,328; 6,451,806; and 6,469,030, each of which is hereby incorporated by reference in its entirety. Such compounds have moderately high molecular weights, a zwitterionic form, and a polarity that prevent penetration of the blood-brain barrier.

Particular piperidine-N-alkylcarbonylates include, for example, N-alkylamino-3,4,4-substituted piperidines, such as alvimopan represented below as formula (IV):

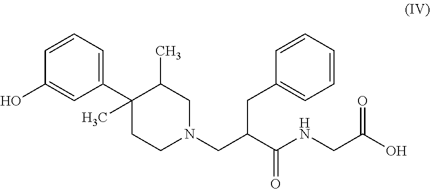 [SMILES: CC1CN(CC(Cc2ccccc2)C(=O)NCC(=O)O)CCC1(C)c1cccc(O)c1]

Alvimopan is available from Adolor Corp., Exton, Pa.

Still other suitable peripheral opioid receptor antagonist compounds may include, for example, quaternary benzomorphan compounds. Quaternary benzomorphan compounds may have the following formula (V):

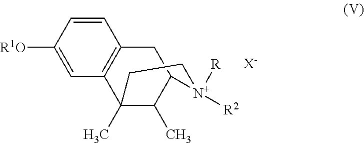

wherein R¹ is hydrogen, acyl, or acetoxy; and R² is alkyl or alkenyl; R is alkyl, alkenyl, or alkynyl and X⁻ is an anion, such as a chloride, bromide, iodide, or methylsulfate anion.

Specific quaternary derivatives of benzomorphan compounds that may be employed in the methods as disclosed herein include, for example, the following compounds of formula (V): 2′-hydroxy-5,9-dimethyl-2,2-diallyl-6,7-benzomorphanium-bromide; 2′-hydroxy-5,9-dimethyl-2-n-propyl-2-allyl-6,7-benzomorphanium-bromide; 2′-hydroxy-5,9-dimethyl-2-n-propyl-2-propargyl-6,7-benzomorphanium-bromide; and 2′-acetoxy-5,9-dimethyl-2-n-propyl-2-allyl-6,7-benzomorphanium-bromide.

Other quaternary benzomorphan compounds that may be employed in methods of the invention are described, for example, in U.S. Pat. No. 3,723,440, the entire disclosure of which is incorporated herein by reference.

Other peripheral opioid antagonists include, for example, 6-carboxy-normorphinan derivatives, particularly N-methyl-C-normorphinan derivatives, as described in U.S. Published Application No. 2008/0064744, which is hereby incorporated by reference in its entirety, and including VI):

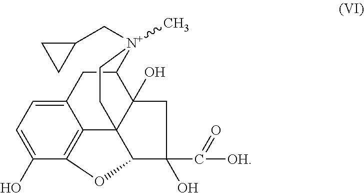

In certain embodiments, opioid receptor antagonists formulated with phosphatidylcholine (PC) are contemplated. Phosphatidylcholines (PC) are a class of phospholipids that is composed of a choline head group and glycerophosphoric acid with a variety of fatty acids which exhibit absorption-enhancing properties. Phosphatidylcholines (PC) are commercially available from, e.g., Lipoid LLC, Newark, N.J. Specific PC formulations include, for example, phosphatidylcholine-formulated methylnaltrexone (MNTX-PC). MNTX may be formulated with the choline head group of PC with an ionic bond or by interaction between ions.

In certain embodiments, the pharmaceutical compositions of the present invention include an opioid receptor antagonist, for example, methylnaltrexone, complexed with phosphatidylcholine. As used herein, such complexes can refer to the interaction of the opioid receptor antagonist and phosphatidylcholine after being dissolved in a solvent and subsequently removing the solvent.

Other peripheral opioid antagonist formulations may include polymer formulations of opioid antagonists, as described in U.S. Published Application No. 2006/0105046, hereby incorporated by reference. Specific polymer formulations include, for example, PEGylated naloxone and naltrexone.

Embodiments also encompass administration of more than one opioid receptor antagonist formulations. Combinations of one or more opioid receptor antagonist formulations with one or more opioid receptor antagonists are also contemplated, for example, a combination of MNTX-PC and alvimopan.

### CHEMICAL DEFINITIONS

“Alkyl” refers to a univalent aliphatic hydrocarbon group which is saturated and which may be straight, branched, or cyclic having from 1 to about 10 carbon atoms in the chain, and all combinations and subcombinations of chains therein. Exemplary alkyl groups include, but are not limited to, methyl, ethyl, n-propyl, isopropyl, butyl, isobutyl, sec-butyl, tert-butyl, pentyl, cyclopropyl, cyclobutyl, cyclopentyl, and cyclohexyl.

“Lower alkyl” refers to an alkyl group having 1 to about 6 carbon atoms.

“Alkenyl” refers to a univalent aliphatic hydrocarbon group containing at least one carbon-carbon double bond and having from 2 to about 10 carbon atoms in the chain, and all combinations and subcombinations of chains therein. Exemplary alkenyl groups include, but are not limited to, vinyl, propenyl, butynyl, pentenyl, hexenyl, and heptnyl.

“Alkynyl” refers to a univalent aliphatic hydrocarbon group containing at least one carbon-carbon triple bond and having from 2 to about 10 carbon atoms in the chain, and combinations and subcombinations of chains therein. Exemplary alkynyl groups include, but are not limited to, ethynyl, propynyl, butynyl, pentynyl, hexynyl, and heptynyl.

“Alkylene” refers to a divalent aliphatic hydrocarbon group having from 1 to about 6 carbon atoms, and all combinations and subcombinations of chains therein. The alkylene group may be straight, branched, or cyclic. There may be optionally inserted along the alkylene group one or more oxygen, sulfur, or optionally substituted nitrogen atoms, wherein the nitrogen substituent is an alkyl group as described previously.

“Alkenylene” refers to a divalent alkylene group containing at least one carbon-carbon double bond, which may be straight, branched, or cyclic. Exemplary alkenylene groups include, but are not limited to, ethenylene (—CH═CH—) and propenylene (—CH═CHCH₂—).

“Cycloalkyl” refers to a saturated monocyclic or bicyclic hydrocarbon ring having from about 3 to about 10 carbons, and all combinations and subcombinations of rings therein. The cycloalkyl group may be optionally substituted with one or more cycloalkyl-group substituents. Exemplary cycloalkyl groups include, but are not limited to, cyclopropyl, cyclobutyl, cyclopentyl, cyclohexyl, and cycloheptyl.

“Acyl” means an alkyl-CO group wherein alkyl is as previously described. Exemplary acyl groups include, but are not limited to, acetyl, propanoyl, 2-methylpropanoyl, butanoyl, and palmitoyl.

“Aryl” refers to an aromatic carbocyclic radical containing from about 6 to about 10 carbons, and all combinations and subcombinations of rings therein. The aryl group may be optionally substituted with one or two or more aryl group substituents. Exemplary aryl groups include, but are not limited to, phenyl and naphthyl.

“Aryl-substituted alkyl” refers to a linear alkyl group, preferably a lower alkyl group, substituted at a terminal carbon with an optionally substituted aryl group, preferably an optionally substituted phenyl ring. Exemplary aryl-substituted alkyl groups include, for example, phenylmethyl, phenylethyl, and 3(4-methylphenyl)propyl.

“Heterocyclic” refers to a monocyclic or multicyclic ring system carbocyclic radical containing from about 4 to about 10 members, and all combinations and subcombinations of rings therein, wherein one or more of the members of the ring is an element other than carbon, for example, nitrogen, oxygen, or sulfur. The heterocyclic group may be aromatic or nonaromatic. Exemplary heterocyclic groups include, for example, pyrrole and piperidine groups.

“Halo” refers to fluoro, chloro, bromo, or iodo.

Compounds employed in the methods as disclosed herein (e.g., opioid receptor antagonists) may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastercomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds as disclosed herein are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The compounds as disclosed herein also encompass their salts. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts, such as alkylammonium salts. Some embodiments contemplate nontoxic, pharmaceutically acceptable salts as described herein, although other salts may be useful, as, for example, in isolation or purification steps. Salts include, but are not limited to, sodium, lithium, potassium, amines, tartrates, citrates, hydrohalides, phosphates and the like.

The compounds employed in the methods as disclosed herein may exist in prodrug form. As used herein, “prodrug” is intended to include any covalently bonded carriers which release the active parent drug or compounds that are metabolized in vivo to an active drug or other compounds employed in the methods of the invention in vivo when such prodrug is administered to a subject. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, embodiments encompass prodrugs of the compounds as disclosed herein as well as methods of delivering prodrugs. Prodrugs of the compounds may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

### Methods of Administration and Other Formulation Considerations

The pharmaceutical compositions as disclosed herein can comprise an effective amount of one or more candidate substances (e.g., a phosphatidylcholine formulations of the present invention) or additional agents dissolved or dispersed in a pharmaceutically acceptable carrier. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it needs to be sterile for such routes of administration. The pharmaceutical compositions of the present invention may be administered orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in creams, in lipid compositions, via a catheter, via a lavage, via continuous infusion, via infusion, via inhalation, via injection, via local delivery, via localized perfusion, bathing target cells directly, or by other method or any combination of the foregoing as would be known to one of ordinary skill in the art (see, for example, Remington's Pharmaceutical Sciences, 1990). In some embodiments, a pharmaceutical composition may be formulated for oral delivery. In certain embodiments, intramuscular, intravenous, topical administration, or inhalation administration is contemplated. In certain embodiments, oral administration is contemplated.

In some embodiments, a pharmaceutical composition of the present invention is administered to a subject using a drug delivery device. Any drug delivery device is contemplated in this regard.

The actual dosage amount of an opioid receptor antagonist formulation comprised in a pharmaceutical composition of the present invention that is administered to a subject can be determined by physical and physiological factors such as body weight, severity of condition, the type of disease being treated, previous or concurrent therapeutic interventions, idiopathy of the patient and on the route of administration. The practitioner responsible for administration will typically determine the concentration of active ingredient(s) in a composition and appropriate dose(s) for the individual subject.

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. The time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, about 2 weeks to about 4 weeks, or longer, or any time interval derivable within any of these recited ranges. For example, the time interval between doses can be about 1 hour, 2 hours, 3 hours, 4 hours, 5 hours, 6 hours, 7 hours, 8 hours, 9 hours, 10 hours, 11 hours, 12 hours, 15 hours, 18 hours, 21 hours, 24 hours, 1 day, 2 days, 3 days, 4 days, 5 days, 6 days, 7 days, 8 days, 9 days, 10 days, 11 days, 12 days, 13 days, 14 days, 3 weeks, 4 weeks or longer.

In certain embodiments, it may be desirable to provide a continuous supply of a pharmaceutical composition to the patient. This can be accomplished by catheterization, followed by continuous administration of the therapeutic agent, for example. The administration can be intra-operative or post-operative.

In certain embodiments, pharmaceutical compositions may comprise, for example, at least about 0.1% (w/w) of an opioid receptor antagonist conjugate. In some embodiments, the pharmaceutical compositions can comprise, for example, from about 0.1% to about 2% (w/w) of an opioid receptor antagonist conjugate. In some embodiments, the opioid receptor antagonist formulation may comprise between about 2% to about 75% of the weight of the unit, or between about 25% to about 60%, for example, and any range derivable therein. In other non-limiting examples, a dose may also comprise from about 10 μg/kg/body weight, 100 μg/kg/body weight, 200 μg/kg/body weight, 350 μg/kg/body weight, 500 μg/kg/body weight, 1 mg/kg/body weight, 2.5 mg/kg/body weight, 5 mg/kg/body weight, 7.5 mg/kg/body weight, 10 mg/kg/body weight, 25 mg/kg/body weight, 50 mg/kg/body weight, 75 mg/kg/body weight, 100 mg/kg/body weight, 125 mg/kg/body weight, 150 mg/kg/body weight, 175 mg/kg/body weight, 200 mg/kg/body weight, 250 mg/kg/body weight, 300 mg/kg/body weight, 350 mg/kg/body weight, 400 mg/kg/body weight, 450 mg/kg/body weight, or 500 mg/kg/body weight to about 1000 mg/kg/body weight or more of the opioid receptor antagonist formulation per administration, or any range derivable therein. In a non-limiting example of a derivable range from the numbers listed herein, a range of about 0.1 mg/kg/body weight to about 20 mg/kg/body weight may be administered.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The opioid receptor antagonist formulation may be formulated into a composition, such as a pharmaceutical composition, in a free base, neutral, or salt form. Pharmaceutically acceptable salts are described herein.

In embodiments wherein a carrier is employed, such a carrier may be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In some embodiments, the composition can include isotonic agents, such as, for example, sugars, sodium chloride, or combinations thereof.

In some embodiments, one may use eye drops, nasal solutions or sprays, aerosols or inhalants containing compositions as disclosed herein. Such compositions are generally designed to be compatible with the target tissue type. In a non-limiting example, nasal solutions can be aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions can be isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. In some embodiments, suspensions and capsules are contemplated. Oral compositions may be incorporated directly with the food of the diet. In certain embodiments, carriers for oral administration comprise inert diluents (e.g., glucose, lactose, or mannitol), assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, or combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Sterile injectable solutions may be prepared by incorporating a particle as disclosed herein in the required amount in the appropriate solvent with various of the other ingredients enumerated above, as required, followed by sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterilized liquid medium thereof. The liquid medium should be suitably buffered if necessary and the liquid diluent (e.g., water) first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

Exemplary subjects who may receive administration of the compositions disclosed herein include those who are on opioid therapy, who have recently been on opioid therapy or who intend to be on opioid therapy. In some embodiments, the subject, at the time of the screening for treatment, is on an opioid therapeutic regimen and has been on such regimen for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days. In some embodiments, the subject has been taking opioids for at least one month. In some embodiments, the subject, at the time of the screening, will begin an opioid therapeutic regimen at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days after the screening. In some embodiments, the subject, at the time of the screening, will have discontinued opioid therapeutic regimen less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80 85, 90, 95 or 100 days prior to the screening.

The subject may be on an opioid regimen for a variety of purposes. For example, the subject may be a cancer or surgical patient, an immunosuppressed or immunocompromised patient (including HIV infected patient), a patient with advanced medical illness, a terminally ill patient, a patient with neuropathies, a patient with rheumatoid arthritis, a patient with osteoarthritis, a patient with chronic pack pain, a patient with spinal cord injury, a patient with chronic abdominal pain, a patient with chronic pancreatic pain, a patient with pelvic perineal pain, a patient with fibromyalgia, a patient with chronic fatigue syndrome, a patient with migraine or tension headaches, a patient on hemodialysis, or a patient with sickle cell anemia. In some embodiments, the subject is receiving opioids for alleviation of pain. In some embodiments, the subject is receiving opioids for alleviation of chronic non-malignant pain. As used herein, the term “non-malignant pain” refers to pain originating from a nonmalignant source such as cancer. In some embodiments, non-malignant pain includes back pain, cervical pain, neck pain, fibromyalgia, low extremity pain, hip pain, migraines, headaches, neuropathic pain, or osteoarthritis.

Embodiments disclosed herein may be of therapeutic value in opioid antagonist treatment for patients who have tumors. Such tumors include, but are not limited to adrenal cortical carcinoma, tumors of the bladder: squamous cell carcinoma, urothelial carcinomas; tumors of the bone: adamantinoma, aneurysmal bone cysts, chondroblastoma, chondroma, chondromyxoid fibroma, chondrosarcoma, fibrous dysplasia of the bone, giant cell tumour, osteochondroma, osteosarcoma; breast tumors: secretory ductal carcinoma, chordoma; colon tumors: colorectal adenocarcinoma; eye tumors: posterior uveal melanoma, fibrogenesis imperfecta ossium, head and neck squamous cell carcinoma; kidney tumors: chromophobe renal cell carcinoma, clear cell renal cell carcinoma, nephroblastoma (Wilms tumor), kidney: papillary renal cell carcinoma, primary renal ASPSCR1-TFE3 tumor, renal cell carcinoma; liver tumors: hepatoblastoma, hepatocellular carcinoma; lung tumors: non-small cell carcinoma, small cell cancer; malignant melanoma of soft parts; nervous system tumors: medulloblastoma, meningioma, neuroblastoma, astrocytic tumors, ependymomas, peripheral nerve sheath tumors, phaeochromocytoma; ovarian tumors: epithelial tumors, germ cell tumors, sex cord-stromal tumors, pericytoma; pituitary adenomas; rhabdoid tumor; skin tumors: cutaneous benign fibrous histiocytomas; smooth muscle tumors: intravenous leiomyomatosis; soft tissue tumors: liposarcoma, myxoid liposarcoma, low grade fibromyxoid sarcoma, leiomyosarcoma, alveolar soft part sarcoma, angiomatoid fibrous histiocytoma (AFH), clear cell sarcoma, desmoplastic small round cell tumor, elastofibroma, Ewing's tumors, extraskeletal myxoid chondrosarcoma, inflammatory myofibroblastic tumor, lipoblastoma, lipoma/benign lipomatous tumors, liposarcoma/malignant lipomatous tumors, malignant myoepithelioma, rhabdomyosarcoma, synovial sarcoma, squamous cell cancer; tumors of the testis: germ cell tumors, spermatocytic seminoma; thyroid tumors: anaplastic (undifferentiated) carcinoma, oncocytic tumors, papillary carcinoma; uterus tumors: carcinoma of the cervix, endometrial carcinoma, leiomyoma etc. The invention also provides a method of treating abnormal tumors, comprising administering to a patient in need of such treatment, an effective amount of an opioid antagonist.

As used herein, the term “chronic” refers to a condition that persists for an extended period of time. In some embodiments, chronic may refer to a condition that lasts at least 1, 2, 3 or 4 weeks. In some embodiments, chronic may refer to a condition that lasts at least 1, 2, 3, 4, 5, 6, 7, 8, 9, 10, 11, 12, 18, 24, 30 or 36 months. In some embodiments, the subject is receiving opioids for alleviation of chronic non-malignant pain that has persisted for at least 2 months.

In some embodiments, the subject may be on opioid therapy including, but not limited to, alfentanil, anileridine, asimadoline, bremazocine, burprenorphine, butorphanol, codeine, dezocine, diacetylmorphine (heroin), dihydrocodeine, diphenoxylate, fedotozine, fentanyl, funaltrexamine, hydrocodone, hydromorphone, levallorphan, levomethadyl acetate, levorphanol, loperamide, meperidine (pethidine), methadone, morphine, morphine-6-glucoronide, nalbuphine, nalorphine, opium, oxycodone, oxymorphone, pentazocine, propiram, propoxyphene, remifentanyl, sufentanil, tilidine, trimebutine, and/or tramadol.

In some embodiments, the subject is receiving a daily dose of at least 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 mg of oral morphine equivalents. In some embodiments, the subject is receiving at least 50 mg of oral morphine equivalents. Calculation of oral morphine equivalents is well known in the art.

The subject's opioid therapeutic regimen may be by any mode of administration. For example, the subject may be taking opioids orally, transdermally, intravenously, or subcutaneously.

### Combination Therapy

In order to enhance or increase the effectiveness of an opioid receptor antagonist formulation comprised in a pharmaceutical composition as disclosed herein, the particle may be combined with another therapy, such as another agent that combats and/or prevents a disorder mediated by opioid receptor activity. For example, a pharmaceutical composition may be provided in a combined amount with an effective amount of a second opioid receptor antagonist formulation, or an opioid receptor antagonist. Additionally, a pharmaceutical composition may be provided in a combined amount with an effective amount of an anti-cancer agent, as described in U.S. Patent Application No. 2006/0258696, PCT Publication No. WO 06/096626, or PCT Publication No. WO 07/053194, each of which is hereby incorporated by reference in its entirety.

The invention also includes the coadministration of the opioid antagonists with agents that are not opioid antagonists, but which are nonetheless useful in treating disorders characterized by unwanted migration or proliferation of endothelial cells. Examples of such agents include anticancer agents, antineovascularization agents (for example, anti-VEGF monoclonal antibody), antidiabetes agents, anti-sickle cell agents, wound healing agents, and anti-endothelial cell proliferative agents.

The invention also includes a method of attenuating tumor progression and metastasis in animal tissues, comprising contacting tumor cells or tissues with a growth-inhibiting amount of an opioid antagonist, and a method of attenuating proliferation of hyperproliferative cells in a subject, comprising administering to the subject at least one opioid antagonist, in an amount which is effective to attenuate proliferation of the hyperproliferative cells. In one embodiment, the method involves administering a peripheral opioid antagonist, and, in particular, a quaternary derivative of noroxymorphone, to a subject with cancer, whether or not the cancer involves angiogenesis, to treat or inhibit the development or recurrence of the cancer. Cancers not involving angiogenesis include those that do not involve the formation of a solid tumor fed by neovasculature. Certain blood cell cancers fall into this category, for example: leukemias (cancer of the leukocytes or white cells), lymphomas (arising in the lymph nodes or lymphocytes), and some cancers of the bone marrow elements. Thus, in one aspect of the invention, a method of treatment is provided. The method involves administering to a subject with a disorder characterized by hyperproliferation of cells an effective amount of a peripheral opioid antagonist. In one embodiment, the cells are cancer cells. The cancer cells may be cancer cells associated with angiogenesis or they may be unassociated with angiogenesis. In one embodiment, the peripheral opioid antagonist is methylnaltrexone.

In further embodiments, the invention provides methods of treating cancer, wherein a peripheral opioid antagonist and at least one other therapeutic agent that is not an opioid or opioid antagonist are co-administered to the patient. Suitable therapeutic agents include anticancer agents (including chemotherapeutic agents and antineoplastic agents), as well as other antiangiogenesis agents. It has been disovered that opioid antagonists co-administered with various anticancer drugs, radiotherapy or other antiangiogenic drugs can give rise to a significantly enhanced antiproliferative effect on cancerous cells, thus providing an increased therapeutic effect, e.g., employing peripheral opioid antagonists to certain tumors can potentiate their response to other therapeutic regimens. Specifically, a significantly increased antiproliferative effect, including but not limited to a significantly increased antiangiogenic effect, is obtained with co-administered combinations as described in more detail below. Not only can an existing regimen be enhanced, but new regimens are possible, resulting, for example, in lower concentrations of the anticancer compound, a lower dosing of radiation, or lower concentration of other antiangiogenic drugs, compared to the treatment regimes in which the drugs or radiation are used alone. There is the potential, therefore, to provide therapy wherein adverse side effects associated with the anticancer or other antiangiogenic drugs or radiotherapy are considerably reduced than normally observed with the anticancer or other antiangiogenic drugs or radiotherapy when used alone. Thus, in one aspect of the invention, a method of treatment is provided. The method involves administering to a subject with a disorder characterized by hyperproliferation of cells an effective amount of an opioid antagonist and an anticancer agent, radiation, or an antiangiogenic agent. In one embodiment, the cells are cancer cells. In one embodiment, the opioid antagonist is a peripheral opioid antagonist. In one embodiment, the peripheral opioid antagonist is methylnaltrexone. In another aspect of the invention, a method of reducing the risk of recurrence of a cancer in a subject after medical intervention is provided. The method involves administering to the subject before, during or after the medical intervention an effective amount of an opioid antagonist and an anticancer agent, radiation, or an antiangiogenic agent. In one embodiment, the opioid antagonist is a peripheral opioid antagonist. In one embodiment, the peripheral opioid antagonist is methylnaltrexone.

It is contemplated that a combination therapy as disclosed herein may be used in vitro or in vivo. These processes may involve administering the agents at the same time or within a period of time wherein separate administration of the substances produces a desired therapeutic benefit. This may be achieved by contacting the cell, tissue, or organism with a composition, such as a pharmaceutically acceptable composition, that includes two or more agents, or by contacting the cell with two or more distinct compositions, wherein one composition includes one agent and the other includes another.

The pharmaceutical composition may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered about 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1, 2, 3, 4, 5, 6, 7 or 8 weeks or more, or any range derivable therein, prior to and/or after administering the candidate substance.

Various combination regimens of the agents may be employed. Non-limiting examples of such combinations are shown below, wherein a pharmaceutical composition of the present invention is “A” and a second agent, such as a second opioid receptor antagonist, is “B”:

A/B/A B/A/B B/B/A A/A/B A/B/B B/A/A A/B/B/B
B/A/B/B B/B/B/A B/B/A/B A/A/B/B A/B/A/B
A/B/B/A B/B/A/A B/A/B/A B/A/A/B A/A/A/B
B/A/A/A A/B/A/A A/A/B/A

### EXAMPLES

The following examples are included to demonstrate certain embodiments. It should be appreciated by those of skill in the art that the techniques disclosed in the examples which follow represent techniques discovered by the inventor to function well in embodiments. However, those of skill in the art should, in light of the present disclosure, appreciate that many changes can be made in the specific embodiments which are disclosed and still obtain a like or similar result without departing from the spirit and scope of embodiments.

Reagents used in each of these examples are commercially available.

### Example 1

### Materials and Methods

Preparation of MNT-PC Formulation.

A phosphatidylcholine-based formulation of MNTX (MNTX-PC) was prepared by dissolving MNTX and PC in ethanol (200 proof). The molar ratio of MNTX and PC was 1:2. The mixture was heated to 60° C. for 2 hours with stirring. Then, the complex was generated by controlled removal of solvent. The residual was dissolved in chloroform, and the chloroform solution was filtered with filter paper. Because MNTX could not have been dissolved in chloroform, the unformulated MNTX could not pass through the filter paper, and was consequently separated with the MNTX-PC. The filtrate, which contained MNTX-PC, was collected and the solvent of the filtrate was evaporated under vacuum, then lyophilized overnight. The solid complex was crushed and a powder form of MNTX-PC was obtained. The effects of solvent, complex ratio and temperature on the formulation efficacy of MNTX in PC were tested. Methanol, ethanol, tetrahydrofuran and chloroform were selected as solvents. Both MNTX and PC were well dissolved in ethanol. When ethanol was used as solvent and the mixing ratio of MNTX to PC was varied, the MNTX's formulation ratio was increased at higher temperature. Subsequent physicochemical assays revealed that MNTX molecularly dispersed in the formation and its chemical structure was not influenced by PC formulation.

Physicochemical Assay of MNTX-PC.

Ultraviolet (UV) analysis was performed on a Shimadzu UV2550 UV-visible spectrophotometer (Shimadzu Corporation, Kyoto, Japan). X-ray diffractometry (XRD) was determined on a D/MAX2500V/PC X-ray diffractometer (Rigaku Americas Corporation, Tokyo, Japan). Monochromatic Cu-Ka radiation was used. The powders of samples were packed tightly in a rectangular aluminum cell before samples were exposed to the X-ray beam. The scanning regions of the diffraction angle, 2θ, were 0-40°. Duplicate measurements were made at ambient temperature. Radiation was detected with a proportional detector.

Animals, MNTX Administration, and Blood Collection.

The experimental protocol was approved by the Institutional Animal Care and Use Committee. Male Sprague-Dawley rats (190-200 g) were obtained from Luye Pharma (Yantai, China) (Wang et al., 2010). Rats were allowed to acclimatize in environmentally controlled quarters (24±2° C. and a 12:12 h light-dark cycle). The rats were fasted for 12 h prior to the experiments.

Ten rats received test compounds via oral gavage. The rats were divided randomly into two groups: MNTX (n=5) and MNTX-PC (n=5). MNTX in a water solution or MNTX-PC were administered at 250 mg/kg. Venous blood samples were drawn from the ocular venous plexus at 0, 10, 20, 30, 45, 60, 90, 120, 150, 180, 240, 300, 420 and 540 min. The samples were placed into heparinized tubes and centrifuged at 1500×g for 5 min. The plasma samples were immediately stored in a freezer (−20° C.) for the pending assay.

Plasma Sample Processing.

A 100 L plasma sample was transferred to a 1.5 mL microcentrifuge tube. Then 0.2 mL of acetonitrile and 0.1 Ctg of ketamine hydrochloride (I.S.) were mixed with the plasma sample and vortexed for 5 min before being centrifuged at 1500×g for 5 min. The supernatant was transferred to another tube and dried under a gentle flow of nitrogen. The residue was dissolved in 100 L of mobile phase, and then centrifuge at 15,000×g for 10 min. Lastly, 5 μL of supernatant was injected into the LC/MS/MS system for analysis.

Determination of MNTX Concentration by LC/MS/MS.

The concentrations of MNTX in rat plasma samples were determined with LC/MS/MS. MNTX plasma levels and the internal standard determined by LC/MS/MS were tested at the time points listed above. The assay was performed using an HPLC system with an Agilent 1100 pump, an Agilent 1100 auto sampler, and a Hanbang C18 column (150 mm×2.1 mm, 5 μm) with a guard column (Wang et al., 2011). For the mobile phase, methanol/water (60/40, containing 0.1% of acetic acid) was pumped at a flow rate of 0.2 mL/min. The samples were stored at 4° C. in the auto sampler before 5 μL was injected into the column. The detector was an API 4000 triple quadruple mass (MS) spectrometer (Applied Biosystems, Foster City, Calif.), and Analyst™ software version 4.1 was used for MS control and spectral processing. Using electrospray ionization (ESI) in positive ion mode, the MS parameters were optimized as follows: heater temperature, 350° C.; ion source voltage, 4000V. Multiple reaction monitoring was used, and the selected single charged precursor-production ion pairs were m/z 356.08→226.95 for MNTX and m/z 237.93→125.05 for ketamine hydrochloride.

Data analysis. Pharmacokinetic data were analyzed by software (Kinetic 4.4; Thermo Electron Co., Waltham. Mass.). All data were expressed as the mean±standard error (S.E.). A one-way ANOVA determined whether the results had statistical significance. The level of statistical significance was set at P<0.05.

### Example 2

### Evaluation of the Physicochemical Characteristics of MNTX-PC

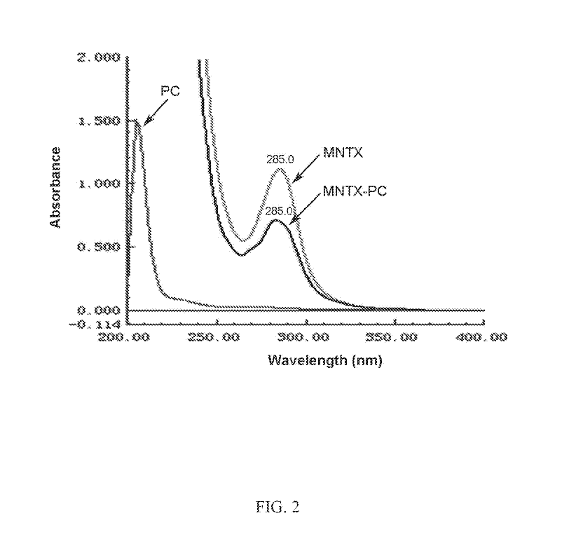

The physicochemical characteristics of MNTX-PC were evaluated with different assays. The UV spectra results are shown in FIG. 2. The characteristic absorption peak of MNTX was present at 285 nm; there was no absorption peak of PC at 285 nm. MNTX-PC had the same absorption peak as MNTX at 285 nm. These results indicate that the prepared MNTX-PC contained MNTX, and MNTX was stable in this formulation.

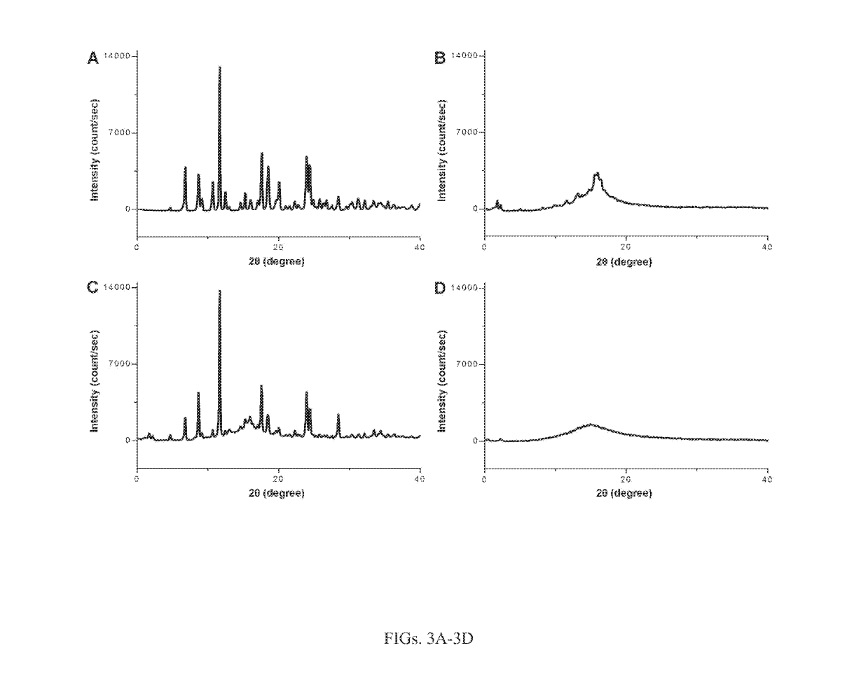

The X-ray diffraction patterns of MNTX, PC, the physical mixture of MNTX and PC, and the formulated MNTX-PC are shown in FIG. 3. The diffraction pattern of MNTX powder displayed sharp crystalline peaks, which is characteristic of an organic molecule with crystallinity. In contrast, PC showed an amorphous form lacking a crystalline peak. For the physical mixture of MNTX and PC, crystalline signals of MNTX were still detected. The crystalline peaks disappeared in MNTX-PC. This result suggests that MNTX in the MNTX-PC formulation was molecularly dispersed.

Liquid chromatography continues to be the most used technique to determine drug concentration in biological matrices (Osinski et al., 2002; Wang et al., 2011). To determine concentrations of MNTX in rat plasma samples, HPLC was coupled with MS/MS to evaluate the bioavailability of MNTX.

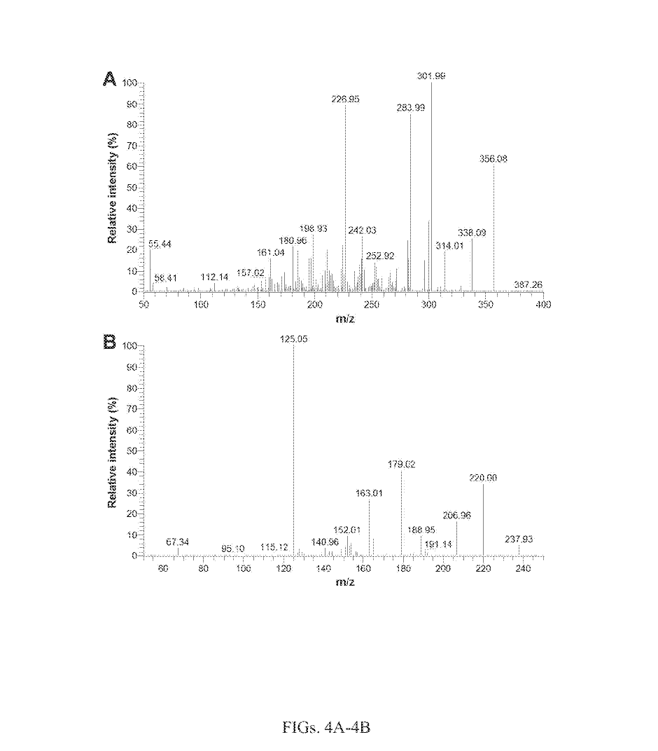

Using electrospray ionization (ESI) in positive ion mode, the molecular ion peaks [M+H]⁺ of MNTX (m/z 356.08) and ketamine hydrochloride (m/z 237.93) were observed. The mass spectra of MNTX and ketamine hydrochloride (internal standard. I.S.) are shown in FIG. 4. For the MS spectrum of MNTX (FIG. 4A), the most abundant fragment ion was that of m/z 301.99, which resulted from the loss of CH₂C(CH₂)₂ from the precursor ion. The calibration curve for MNTX showed good linearity (the correlation coefficient R²: 0.9965) in the concentration range of 10-10,000 ng/mL.

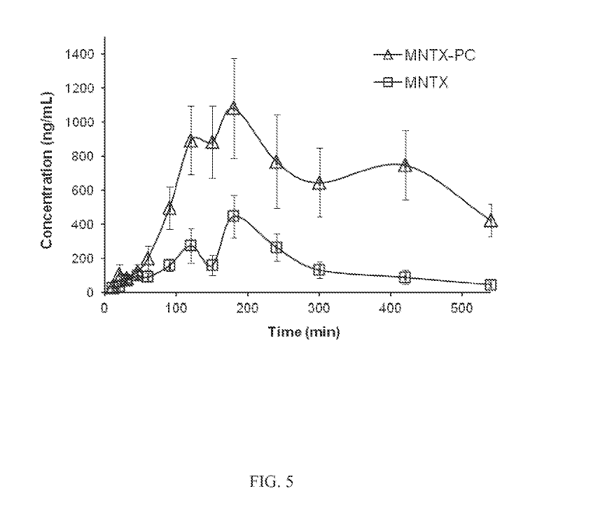

Plasma concentrations of MNTX were compared in the MNTX water solution and MNTX-PC (FIG. 5). After oral administration of 250 mg/kg of MNTX water solution, two plasma MNTX peaks were observed. The Tₘₐₓ of the two peaks was 120 and 180 min. Similar results were also observed in the previous study. For MNTX-PC group, in addition to these two peaks, a third peak (Tₘₐₓ at 420 min) was also observed.

For the MNTX-PC group, the time to peak plasma concentration (Tₘₐₓ) was 180 min, the peak plasma concentration (Cₘₐₓ) was 1083.7±293.9 ng/mL, and plasma elimination half-life (T_{1/2}) was 496 min. Corresponding results for the MNTX control group were 180 min, 448.4±126.0 ng/mL and 259 min, respectively.

FIG. 5 also shows two MNTX concentration peaks after oral administration of the MNTX control and MNTX-PC. The third MNTX peak was observed only after administration of MNTX-PC. As shown in Table 1, for the first peak, the Cₘₐₓ and the area under the plasma concentration-time curve (AUC) from 0 to 150 min for MNTX and MNTX-PC were 275.5±101.9 ng/mL, 341.0±94.5 ng·h/mL, and 894.6±203.0 ng/mL, 1,064.1±261.4 ng·h/mL, respectively (both P<0.01). For the second peak, Cₘₐₓ and AUC from 150 to 540 min for MNTX and MNTX-PC were 448.4±126.0 ng/mL, 1,064.9±353.4 ng·h/mL, and 1,083.7±293.9 ng/mL, 4,694.1±1,214.3 ng·h/mL, respectively (both P<0.01). For both MNTX control and MNTX-PC, the second peak was much higher than the first peak. At each plasma time point measured, the MNTX concentration of MNTX-PC was always much higher than that of MNTX control suggesting that the MNTX-PC formulation remarkably enhanced oral absorption.

The plasma level profile, from 0 to 540 min, reflects the overall bioavailability of MNTX and MNTX-PC (FIG. 5). The AUC_{0-540 min} for MNTX-PC was 5758.2±1474.2. ng·h/mL; for MNTX, 1405.9±447.8 ng·h/mL. The relative bioavailability after oral administration of MNTX-PC was 410% compared to that of control (P<0.01). This result demonstrates that the formulated MNTX-PC significantly increased the bioavailability of MNTX.

TABLE 1
Pharmacokinetic parameters in rats treated with 250 mg/kg unformulated MNTX
in water solution (n = 5) or 250 mg/kg oral MNTX-PC (n = 5).

| | | First peak | | | Second peak | | |
|---|---|---|---|---|---|---|---|
| | | Cₘₐₓ | Tₘₐₓ | AUC_{0-150 min} | Cₘₐₓ | Tₘₐₓ | AUC_{150-540 min} |
| | | (ng/mL) | (min) | (ng · h/mL) | (ng/mL) | (min) | (ng · h/mL) |
| MNTX | Mean | 275.5 | 120 | 341.0 | 448.4 | 180 | 1064.9 |
| | S.E. | 101.9 | — | 94.5 | 126.0 | — | 353.4 |
| MNTX-PC | Mean | 894.6 | 120 | 1064.1 | 1083.7 | 180 | 4694.1 |
| | S.E. | 203.0 | — | 261.4 | 293.9 | — | 1322.1 |

Cₘₐₓ, peak plasma concentration; Tₘₐₓ, time to peak plasma concentration; AUC, area under the plasma concentration-time curve.

### REFERENCES

The following references, to the extent that they provide exemplary procedural or other details supplementary to those set forth herein, are specifically incorporated herein by reference.
U.S. Pat. No. 3,723,440
U.S. Pat. No. 4,176,186
U.S. Pat. No. 4,719,215
U.S. Pat. No. 4,861,781
U.S. Pat. No. 5,102,887
U.S. Pat. No. 5,270,328
U.S. Pat. No. 5,972,954
U.S. Pat. No. 6,274,591
U.S. Pat. No. 6,451,806
U.S. Pat. No. 6,469,030
U.S. Published Appl. 2002/0028825
U.S. Published Appl. 2003/0022909
U.S. Published Appl. 2006/0105046
U.S. Published Appl. 2006/0258696
U.S. Published Appl. 2008/0064744
Brown and Goldberg, Neuropharmacology, 24:181-191, 1985. drugsatfda_docs/nda/2008/021964s000_ClinPharmR.pdf on Feb. 2, 2012.
FDA/CDER-Clinical Pharmacology and Biopharmaceutics Review(s) [Methyl-Naltrexone], 2008. Accessed at http://www.accessdata.fda.gov
Glare and Lickiss, J. Pain Symptom Manage, 7:369-371, 1992.
Handbook of Pharmaceutical Salts: Properties, Selection and Use (Stahl and Wermuth, Eds.), Verlag Helvetica Chimica Acta, 2002.
Klepstad et al., Acta Anaesthesiol. Scand., 44:656-664, 2000.
Kurz and Sessler, Drugs, 63:649-671, 2003.
Mehendale and Yuan, Dig. Dis., 24:105-112, 2006.
Mercadante et al., Support Care Cancer, 8:307-310, 2000.
Michna et al., J Pain, 12:554-562, 2011.
Osinski et al., J. Chromatogr. B, 780:251-259, 2002.
PCT Appln. WO 06/096626
PCT Appln. WO 07/053194
PCT Appln. WO 98/25613
PCT Appln. WO 99/22737
Remington's Pharmaceutical Sciences, 18th Ed. Mack Printing Company, 1289-1329, 1990.
Rotshteyn et al., Expert Opin. Drug Metab. Toxicol., 7:227-235, 2011.
Russell et al., Eur. J. Pharmacol., 78:255-261, 1982.
Thomas et al., N. Engl. J. Med., 358:2332-2343, 2008.
Walsh, Pain, 18:1-11, 1984.
Wang et al., Am. J. Chin. Med., 38:895-907, 2010.
Wang et al., Am. J. Chin. Med., 39:1161-71, 2011.
Wang et al., Ann. Intern. Med., 155:279-280, 2011.
Warfield, Cancer, 82:2299-2306, 1998.
Yap and Pappagallo, In: The epidemiology of opioid bowel dysfunction. Yun (Ed.), NY, Haworth Medical Press, 61-67, 2005.
Yuan and Foss, JAMA, 284:1383-1384, 2000.
Yuan et al., Clin. Pharmnnacol. Ther., 61:467-475, 1997
Yuan, Ann. Pharmacother., 41:984-993, 2007.

1. A pharmaceutical composition comprising methylnaltrexone (MNTX) and phosphatidylcholine (PC).

2. The pharmaceutical composition of claim 1, wherein the composition comprises a complex of methylnaltrexone and phosphatidylcholine.

3.-4. (canceled)

5. The pharmaceutical composition of claim 4, wherein the orally administrable pharmaceutical composition is comprised in a suspension or a capsule.

6.-7. (canceled)

8. The pharmaceutical composition of claim 2, wherein the composition comprises a lyophilized complex of methylnaltrexone and phosphatidylcholine (PC).

9. A method of making the composition of claim 1, comprising:
(a) dissolving MNTX and phosphatidylcholine (PC) in a solvent to form a mixture;
(b) heating the mixture;
(c) removing the solvent to obtain a residual; and
(d) lyophilizing the residual to form a solid substance of phosphatidylcholine (PC)-based MNTX.

10. The method of claim 9, further comprising (i) dissolving the residue in a second solvent prior to lyophilizing the residual.

11. The method of claim 10, further comprising (ii) removing the second solvent to obtain a second residual prior to lyophilizing the residual.

12.-13. (canceled)

14. The method of claim 10, wherein the second solvent is selected from the group consisting of methanol, ethanol, tetrahydrofuran and chloroform.

15.-18. (canceled)

19. The method of claim 9, wherein the molar ratio of methylnaltrexone (MNTX) and phosphatidylcholine (PC) is from 2:1 to 1:10.

20. The method of claim 19, wherein the molar ratio methylnaltrexone (MNTX) and phosphatidylcholine (PC) is from 1:1 to 1:5.

21.-22. (canceled)

23. A method comprising administering a pharmaceutical composition comprising MNTX formulation and a pharmaceutically acceptable carrier to a patient, wherein the MNTX is formulated with phosphatidylcholine (PC).

24. The method of claim 23, wherein the administration is orally, intraadiposally, intraarterially, intraarticularly, intradermally, intralesionally, intramuscularly, intranasally, intraocularally, intraperitoneally, intrapleurally, intrarectally, intrathecally, intratracheally, intraumbilically, intravenously, intravesicularly, intravitreally, liposomally, locally, mucosally, parenterally, rectally, subconjunctival, subcutaneously, sublingually, topically, transbuccally, transdermally, in creams, in lipid compositions, via a catheter, via a lavage, via continuous infusion, via infusion, via inhalation, via injection, via local delivery, via localized perfusion, bathing target cells directly, or any combination thereof.

25. The method of claim 24, wherein the administration is orally, intravenously, or via injection.

26. (canceled)

27. The method of claim 23, wherein the administering comprises administering a dosage of PC-formulated MNTX that ranges from about 0.1-50 mg/kg.

28. The method of claim 27, wherein the administering comprises administering a dosage of PC-formulated MNTX that ranges from about 0.5-5 mg/kg.

29. The method of claim 28, wherein the administering comprises administering a dosage of PC-formulated MNTX that is about 2 mg/kg.

30. The method of claim 23, wherein the patient is suffering from or is at risk of suffering from constipation, dysphoria, pruritus, or urinary retention.

31. The method of claim 23, wherein the patient is suffering from or is at risk of suffering a disorder selected from ileus, post-operative ileus, paralytic ileus, post-partum ileus, gastrointestinal dysfunction developing following abdominal surgery, and idiopathic constipation.

32. The method of claim 23, wherein the patient is suffering from a disorder mediated by opioid receptor activity selected from cancer involving angiogenesis, an inflammatory disorder, immune suppression, a cardiovascular disorder, chronic inflammation, chronic pain, sickle cell anemia, a vascular wound, retinopathy, decreased biliary secretion, decreased pancreatic secretion, biliary spasm, and increased gastroesophageal reflux.

33.-35. (canceled)

36. The method of claim 23, wherein the patient is suffering from an opioid induced side effect wherein the opioid induced side effect comprises at least one effect selected from inhibition of intestinal motility, gastrointestinal dysfunction, constipation, bowel hypomotility, impaction, gastric hypomotility, inhibition of gastric motility, inhibition of gastric emptying, delayed gastric emptying, incomplete evacuation, nausea, emesis, cutaneous flushing, bloating, abdominal distension, sweating, dysphoria, pruritis, and urinary retention.

37.-87. (canceled)
